(19) United States
(12) Patent Application Publication
(10) Pub. No.: US 2003/0099952 A1
(43) Pub. Date: May 29, 2003

(54) Microarrays with visible pattern detection

(76) Inventors: Roland Green, Madison, WI (US); Thomas Albert, Madison, WI (US); Emile Nuwaysir, Madison, WI (US)

Correspondence Address: Nicholas J. Seay, Quarles & Brady LLP, 1 South Pinckney Street, P O Box 2113, Madison, WI, 53701-2113, US

(21) Appl. No.: 09/994,145

(22) Filed: Nov. 26, 2001

(51) Int. Cl. C12Q 1/68; C12M 1/34; B05D 3/00

(52) U.S. Cl. 435/6; 435/287.2; 427/2.11

(57) ABSTRACT

A polynucleotide or polypeptide microarray that contains a set of polynucleotide or polypeptide features including probes for detecting an event or condition of interest is disclosed. The features include some intended to be positive controls. The set of probes are arranged on the microarray in a pattern that is recognizable to a human being through visual observation. If the event or condition of interest has occurred, this can be determined by hybridizing nucleic acids from a sample to the microarray and observing the presence or absence of the visual pattern. A method of building and a method of using the above microarray are also disclosed.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

A DNA microarray is an array, typically on a planar substrate, of a plurality of cells or features each of which contains a set of single stranded DNA probes of unique cell-specific sequence. The advent of modem DNA microarray technology makes it possible to build arrays containing hundreds to hundreds of thousands of features, each containing single stranded DNA probes of different sequence, in a very small area and to perform nucleic acids analysis on biological samples using all these sequences at once. The DNA microarray technology has been applied to many areas such as gene expression and discovery, mutation detection, allelic and evolutionary sequence comparison, genome mapping, and more. A huge amount of data can be generated in a study involving DNA microarrays and thus the largest logistic burden in some studies using microarray can actually be data analysis rather than the data gathering.

DNA microarrays can be constructed by several methods. The arrays of the highest density of features are made by in situ synthesis of DNA probes. One method involves the creation of fixed photolithographic masks which are used to screen light to the array under construction so that nucleotides are added to the array under construction in the areas where light impinges upon the array. Such a method is described in U.S. Pat. No.5,143,854, among others. While this method is efficient and proven, a drawback of the method is that it requires the manufacture of many masks for each microarray to be synthesized and thus is not well adapted to the construction of custom microarrays. A more flexible approach to microarray synthesis is presented in PCT Application No. 99/42813, in which light is used to build probes in the array, but no masks are used. Instead, this technique uses a micromirror device operating under computer control to selectively direct light to or away from cells of the array under construction. Using this approach, the sequence of the probes in an array is determined under software control, and thus is inherently flexible, thus permitting the microarray technology to be used for performing unique and custom experiments.

Both custom and standard DNA arrays are now widely used in scientific studies and experiments. As is often the case for experiments involving biological systems, experiments in a study involving DNA microarrays may or may not work properly for a variety of reasons, some of which may be completely obscure. The potential for resource wastage can be large when a great deal of time and resources have been invested in analyzing the data from a microarray experiment only to find out that the underlying experiment did not work as intended.

### BRIEF SUMMARY OF THE INVENTION

The present invention provides a polynucleotide or polypeptide microarray that contains a set of polynucleotide or polypeptide probes for detecting an event of interest. The probes are arranged on the microarray such that positive controls cells or features are arranged on the array in a pattern that can be recognized by a human being through visual observation. Recognizing such a pattern allows the experimenter to determine whether the event of interest has occurred. The results can be tested for experimental validity simply by hybridizing molecules from a source to the microarray and observing the presence or absence of the pattern.

The present invention also provides a method for building a microarray by selecting and positioning the set of probes on a microarray substrate so that positive control probes will, if hybridized to a complementary nucleic acid that is visually perceptible, will form a visually perceptible pattern. In addition, the present invention provides a method for detecting whether an event of interest has occurred by providing the above microarray, hybridizing molecules from a source to the microarray, and observing the presence or absence of the pattern.

It is an object of the present invention to provide a simple tool to evaluate whether one or more steps of a study involving a microarray has been carried out successfully.

It is an advantage of the present invention that the success of one or more steps of a study involving a microarray can be assessed quickly before a sophisticated and time consuming data analysis is conducted.

Other objects, features and advantages of the present invention will become apparent upon consideration of the following detailed description taken in conjunction with the accompanying drawing.

### BRIEF DESCRIPTION OF THE DRAWING

FIG. 1 depicts five probes arranged in a check pattern which is recognizable to a human being through visual observation.

### DETAILED DESCRIPTION OF THE INVENTION

The present invention is directed at arranging at least one set of polynucleotide probes on a DNA microarray, selected for detecting an event of interest, in a pattern that can be recognized by a human being through visual observation. Such an arrangement allows the occurrence of the event be determined through visually observing the presence or absence of the pattern on the microarray after hybridization reactions. When the microarray is used to study the occurrence of the event, the present invention provides an easy way of obtaining results. When the microarray is used to study a subject that requires further data analysis, a decision on whether to proceed with the analysis can be made based on the presence and absence of the pattern, which is especially helpful when the data analysis is very complicated and time consuming. It is preferred that the probes in the cells forming the visually perceptible pattern are positive controls, i.e. probes expected to hybridize, thus allowing the visual pattern to indicate that the sampling or underlying experiment has been done correctly.

The typical method current in the art to use DNA microarrays is to collect the nucleic acid as samples from an experiment or test and then label the nucleic acids samples with a florescent marker. The nucleic acids can be DNA, for genetic tests, or can be RNA, for gene expression analysis. Sometimes two different florescent markers are used so that two different nucleic acid samples can be tested with the same microarray. The labeled single stranded nucleic acids are then exposed to the microarray so that the sample nucleic acids will hybridize to the single stranded DNA probes on the microarray at those locations where the sequence of the sample nucleic acids and the probes are complementary. The array is washed and then the array is illuminated such that the features where a hybridization event has occurred will emit light. The arrays are typically read by automated readers which record the amount of light emitted from a cell or features, this being an indication of whether or not the complementary nucleic acids is in the sample.

In accordance with the precepts of the scientific method, it is common to incorporate into a microarray some probes which are intended to be positive controls. By positive controls it is meant to refer to probes which should hybridize to nucleic acids in the sample if things are working as they are supposed to. The positive controls are to indicate the existence of an event or condition that was supposed to have occurred or be true. Positive controls can be of several kinds. If the test being performed is to determine sequence differences in DNA among humans, the positive controls might be sequences of DNA highly conserved in humans so that a match would be found in any human DNA sample. If the test is being performed using a test condition, the positive control might indicate that the condition actually existed and had the desired effect. If the effect of cellular development on gene expression in a cell or tissues is being tested, the positive control might be the RNA of a gene which is known to express in all cells of that organism. If the test being performed is genomic mapping, the positive controls might be common repetitive DNA sequence commonly found in the genome of organisms.

The concept of the present invention is that, particularly for a microarray made under computer control, the individual features on the array can easily be arranged in any desired pattern on the array. It is taught here that at least some of the positive controls can be arranges on the microarray so as to create a human perceptible pattern if they hybridize to nucleic acids in the sample. In other words, if the experiment works, or if the sample comes from the expected organism, of if any other positively defined condition exists, the florescence of the positive controls can be used to create a visual indication that things are as expected. Once the hybridization has been performed, the microarray can simply be visually examined under a visual microscope to see if the expected pattern is present. If the pattern is present, the automated scanning of the array for data collection can proceed. If the pattern is not present, that should indicate some flaw in the sample collection or the underlying experiment that would indicate that any data collected would likely be meaningless in any event. In this way, a rapid and early indication of the validity of the data can be achieved.

The pattern described above can be any pattern that is recognizable to a human being through visual observation. A pattern recognizable to a human being through visual observation means a pattern the presence or the absence of which can be readily determined by a human being after viewing the area where the pattern is located. Examples of a pattern recognizable to a human being through visual observation include, but are not limited to, a letter or a word, a shape such as a straight line, a circle or a triangle, and a symbol such as a check. Depending on the hybridization detection method and the specific arrangement of a pattern, the object being viewed can be the microarray slide itself, a scan image of the slide, or an enlarged scan image of the slide. For example, if the pattern is large enough and the hybridization detection method is the development of florescence or a color, the slide itself can be viewed. Otherwise, scanning or other aids for viewing may be necessary.

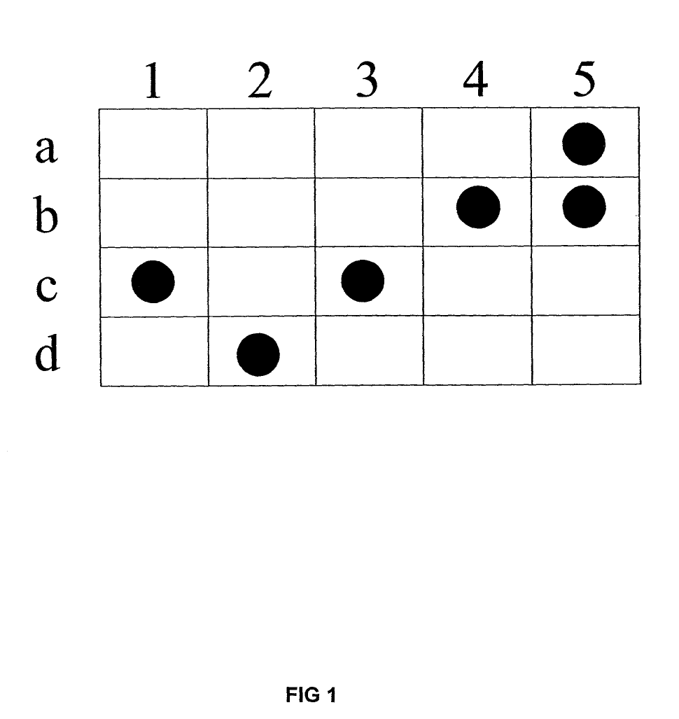

When the probes are arranged to form a pattern in an isolated region on a microarray where no other probes are located at adjacent pixels, the observation of the pattern is free of any interference. When other probes are present at adjacent pixels, the observation of the pattern may be interfered. However, as long as the pattern is still recognizable in spite of the interference, the microarray is within the scope of the present invention. As illustrated in FIG. 1, five probes selected for detecting an event of interest are arranged to locate at pixels 1c, 2d, 3c, 4b and 5a to form a check symbol. In a situation where hybridization occurs at, in addition to the above pixels, pixel 5b as well, the check symbol is nevertheless still recognizable and thus the microarray is within the scope of the present invention.

The choice of patterns is affected by the number of cells or features that are being used to form the pattern. The term feature is used to refer to an area on the array in which the DNA probes have a common sequence which differs from the probes in other features. For visual examination purposes, the features serve as pixels in an image. For example, in the trivial example of using one feature to form a pattern, the pattern is limited to a dot, since there is only one pixel. If five different features are used to form a pattern, the pattern can vary. A thirty five feature area could be used to make any character, number or symbol in the ASCII character set. Since DNA microarrays can have many thousands of possible features, the use of a any reasonable number of features to make such a pattern does not use any significant portion of the total resources of the microarray.

The present invention has many applications. The event of interest may vary according to individual applications. For example, in any application involving microarrays, one may wish to know that hybridization reactions involving the microarrays have worked properly before proceeding with further data analysis. In this case, the event of interest is the hybridization procedure and the set of probes forming the pattern are positive control probes demonstrating the existence of any hybridization reactions.

In another example, microarrays are used to study gene expression changes in response to heat exposure in Saccharomyces cerevisiae. A microarray containing virtually every gene of S. cerevisiae is built first. Then, a group of S. cerevisiae cells are exposed to heat and cDNAs from these cells are prepared and hybridized to the microarray. Before conducting a comprehensive analysis of the data, one may wish to confirm that the cDNAs used in the hybridization are indeed from cells that have been exposed to heat. In this case, the event of interest is heat exposure of the S. cerevisiae cells. DNA sequences from S. cerevisiae genes that are known to be turned on by heat exposure are used as probes to form a pattern on the microarray for determining whether the event of interest has occurred.

For the purpose of the present invention, an event of interest is defined broadly. For example, in the S. cerevisiae example described above, the event of interest can be a combination of heat exposure and proper hybridization. In fact, if the prearranged pattern is observed, the occurrence of both is confirmed. Of course one can also form two patterns on the microarray, one for each event.

In another example, the activation of several signal transduction pathways in S. cerevisiae during heat exposure is studied. In this case, the event of interest is the activation of one or more of the signal transduction pathways. Selected DNA sequences from genes in each pathway that are activated when the pathway is activated are used to form a pattern on a microarray. Such a microarray allows quick identification of the activation of a signal transduction pathway.

So far, we have described building a polynucleotide microarray containing a visible pattern. It is understood that one can similarly build other types of microarrays such as polypeptide microarrays, which can be used to study protein-protein interaction, ligand-receptor interaction and so on.

One of ordinary skill in the art knows how to select probes for a certain application and how to build a microarray with those probes. There are several ways a microarray can be built. One way is to synthesize the probes on a microarray substrate in situ and another way is to synthesize a series of probes and then place them on a microarray substrate (spotting). The exact way a microarray is built is not critical for the present invention. Examples of building a polynucleotide microarray can be found in PCT Patent Publication Nos. WO 99/42813, 92/10092 and 90/15070, U.S. Pat. No. 5,143,854, each of which is hereby incorporated by reference in its entirety. An example of building polypeptide microarrays can be found in Pirrung et al., U.S. Pat. No. 5,143,854 (see also PCT Application No. WO 90/15070), which is also incorporated hereby by reference in its entirety.

We claim:

1. A DNA microarray comprising:
a set of features on a substrate, each of the features including single DNA probes, the probes for positive controls being arranged in a pattern recognizable to a human being through visual observation so that whether an event of interest has occurred can be determined by hybridizing nucleic acids from a sample to the microarray and observing the presence or absence of the visual pattern.

2. The polynucleotide microarray of claim 1, wherein the set of features contains one feature.

3. The polynucleotide microarray of claim 1, wherein the set of features contains a plurality of features.

4. A method for building a polynucleotide microarray comprising the steps of:
selecting a set of features, each feature including polynucleotide probes for detecting an event of interest, some of the features being positive controls; and
arranging the set of features on a microarray substrate so that the positive controls form a pattern recognizable to a human being through visual observation if the positive control features fluoresce.

5. The method of claim 4, wherein the set of features contains one feature.

6. The method of claim 4, wherein the set of features contains a plurality of features.

7. A method for detecting whether an event of interest has occurred comprising the steps of:
providing a DNA microarray comprising a set of features each including single stranded DNA probes for detecting the event of interest, the microarray including features intended to serve as positive controls, the features for positive controls being arranged in a pattern recognizable to a human being through visual observation;
hybridizing nucleic acids from a sample to the microarray; and
observing the presence or absence of the visual pattern.

8. The method of claim 7, wherein the set of features contains one feature.

9. The method of claim 7, wherein the set of features contains a plurality of features.
